(19) United States
(12) Patent Application Publication
(10) Pub. No.: US 2004/0161785 A1
(43) Pub. Date: Aug. 19, 2004

(54) High throughput screening method and system

(76) Inventors: James Norman Cawse, Pittsfield, MA (US); Robert Marcel Mattheyses, Schenectady, NY (US); Carl Harold Hansen, Latham, NY (US); Thomas Robert Kiehl, Troy, NY (US)

Correspondence Address: Philip D. Freedman, Philip D. Freedman PC, P.O. Box 19076, Alexandria, VA, 22320, US

(21) Appl. No.: 10/762,326

(22) Filed: Jan. 23, 2004

Related U.S. Application Data
(62) Division of application No. 09/595,005, filed on Jun. 16, 2000.
(60) Provisional application No. 60/202,747, filed on May 8, 2000.

(51) Int. Cl. C12Q 1/68; G06F 19/00; G01N 33/48; G01N 33/50

(52) U.S. Cl. 435/6; 702/20

(57) ABSTRACT

In an experimental design strategy for evaluating systems with complex physical, chemical and structural requirements, a first population of entities is synthesized, a property of each of the entities can be detected by a high throughput screening (HTS) method and a genetic algorithm based on the property of the entities is executed to identify a second population of entities. A system for screening constructs to determine a problem solution includes a generator to provide a binary string representing a random first population of the constructs, a combinatorial reactor to synthesize the first population of constructs and to determine a fitness function for each construct of the population by a high throughput screening process and an executor to execute a genetic algorithm on the first population to produce a generation that defines a second population of the materials.

### CROSS REFERENCE TO RELATED APPLICATIONS

This application claims priority to and the benefit of the filing date of Provisional Application Serial No. 60/202,747, filed May 8, 2000, entitled “GENETIC ALGORITHMS FOR COMBINATORIAL CHEMISTRY”.

### BACKGROUND

1. Field of the Invention

The present invention relates to a high throughput screening (HTS) method and system.

2. Discussion of Related Art

In experimental reaction systems, each potential combination of reactant, catalyst and condition should be evaluated in a manner that provides correlation to performance in a production scale reactor. Combinatorial organic synthesis (COS) and high throughput screening (HTS) methodology were developed in the pharmaceutical industry approximately 20 years ago. COS uses systematic and repetitive synthesis to produce diverse molecular entities formed from sets of chemical “building blocks.” As with traditional research, COS relies on experimental synthesis methodology. However instead of synthesizing a single compound, COS exploits automation and miniaturization to produce large libraries of compounds sometimes through successive stages, each of which produces a chemical modification of an existing molecule of a preceding stage. The procedure provides large libraries of diverse compounds that can be screened for various activities.

The techniques used to prepare such libraries have typically involved a stepwise or sequential coupling of building blocks to form the compounds of interest. For example, Pirrung et al., U.S. Pat. No. 5,143,854 discloses a technique for generating arrays of peptides and other molecules using, for example, light-directed, spatially-addressable synthesis techniques. Pirrung et al. synthesizes polypeptide arrays on a substrate by attaching photoremovable groups to the surface of the substrate, exposing selected regions of the substrate to light to activate those regions, attaching an amino acid monomer with a photoremovable group to the activated region, and repeating the steps of activation and attachment until polypeptides of the desired length and sequences are synthesized.

Materials development requires investigation of a number of physical, chemical and structural requirements. The number of possible combinations of these requirements may be enormous. For example, in a relatively simple single-phase homogeneous catalyst system, the number of possible experiments can be in the millions. TABLE 1 shows parameters for the design of a homogeneous catalyst system.

TABLE 1

| Formulation Factors | Type | Levels |
|---|---|---|
| Primary Catalyst | Qualitative | 1 |
| Inorganic Cocatalyst | Qualitative | 20 |
| Amount of Cocatalyst | Quantitative | 3 |
| Organic Ligand | Qualitative | 20 |
| Amount of Ligand | Quantitative | 3 |
| Active Anion | Qualitative | 10 |
| Amount of Anion | Quantitative | 3 |
| Process Factors | | |
| Reaction Time | Quantitative | 3 |
| Reaction Temperature | Quantitative | 3 |
| Reaction Pressure | Quantitative | 3 |
| Total Number of Potential Runs | | 2,916,000 |

Of course, multiple phase systems can involve more combinations. It would be extremely difficult for HTS methodology to fully investigate such systems because of the extent of the library combinations. As such, there remains a long-felt a need for a methodology to generate meaningful HTS libraries for systems such as materials systems with complex physical, chemical and structural requirements.

### SUMMARY OF THE INVENTION

Accordingly, the present invention relates to an experimental design strategy for evaluating systems with complex physical, chemical and structural requirements by HTS methodology. In one exemplary embodiment, a first population of entities is synthesized and a property of each of the entities is detected by a high throughput screening (HTS) method. A genetic algorithm based on the property of the entities is executed to identify a second population of entities.

In another embodiment, a high throughput screening (HTS) method comprises (A) depositing each of a first population of entities in respective wells of an array, (B) reacting the population to form a plurality of products, (C) detecting a property of each of the plurality of products and (D) executing a genetic algorithm based on the property of the plurality of products to identify a second population of entities.

In still another embodiment, a method of selecting a carbonylation catalyst is provided. In the method, a first population of prospective carbonylation catalyst entities is synthesized and a property of each of the entities is detected. A genetic algorithm based on the property of the entities is then executed to identify a second population of prospective carbonylation catalyst entities.

A further alternative embodiment of the invention relates to a system for screening constructs to determine a problem solution. The system comprises a generator to provide a binary string representing a random first population of the constructs, a combinatorial reactor to synthesize the first population of constructs and to determine a fitness function for each construct of the population by a high throughput screening process and an executor to execute a genetic algorithm on the first population to produce a generation that defines a second population of the materials.

### BRIEF DESCRIPTION OF THE DRAWINGS

FIG. 1 is a schematic representation of an aspect of an embodiment of the present invention;

FIG. 2 is a schematic representation of an aspect of an embodiment of the present invention; and

FIG. 3 is a graph of experimental points from a genetic algorithmic high throughput screening method.

### DETAILED DESCRIPTION OF PREFERRED EMBODIMENTS

In nature, a gene is the basic functional unit by which hereditary information is passed from parents to offspring. Genes appear at particular places (called gene “loci”) along molecules of deoxyribose nucleic acid (DNA). DNA is a long thread-like biological molecule that has the ability to carry hereditary information and the ability to serve as a model for the production of replicas of itself. All known life forms (including bacteria, fungi, plants, animals and human) are based on the DNA molecule.

The so-called “genetic code” involving the DNA molecule consists of long strings (sequences) of 4 possible molecular values that can appear at the various gene loci along the DNA molecule. The 4 possible molecular values are “bases” named adenine, guanine, cytosine and thymine (abbreviated as A, G, C, and T, respectively). Thus, the “genetic code” in DNA consists of a long string such as CTCGACGGT . . . .

Genetic algorithms are search algorithms based on the mechanics of natural selection and natural genetics. They combine survival of the fittest among string structures with a structured yet randomized information exchange to form a search algorithm with some of the innovative flair of human search. In every generation, a new set of artificial entities (strings) is created using bits and pieces of the fittest of the old. Randomized genetic algorithms have been shown to efficiently exploit historical information to speculate on new search points with improved performance.

It is contemplated that Genetic algorithms are useful (1) to abstract and rigorously explain adaptive processes of natural systems and (2) to design artificial systems software that would retain important mechanisms of natural systems. This approach has led to important discoveries in both natural and artificial systems science

Typically, the central theme of research on genetic algorithms is robustness, the balance between efficiency and efficacy necessary for survival in different environments. The implications of robustness for artificial systems are manifold. If artificial systems are made more robust, costly redesigns can be reduced or eliminated. If higher levels of adaptation can be achieved, existing systems will perform their functions longer and better.

Genetic algorithms are computer programs that solve search or optimization problems by simulating the process of evolution by natural selection. Regardless of the exact nature of the problem being solved, a typical genetic algorithm cycles through a series of steps that can be as follows:

(1) Initialization: A population of potential solutions is generated. “Solutions” are discrete pieces of data that have the general shape (e.g., the same number of variables) as the answer to the problem being solved. For example, if the problem being considered is to find the best six coefficients to be plugged into a large empirical equation, each solution will be in the form of a set of six numbers, or in other words a 1×6 matrix or linked list. These solutions can be easily handled by a digital computer.

(2) Rating: A problem-specific evaluation function is applied to each solution in the population, so that the relative acceptability of the various solutions can be assessed.

(3) Selection of parents: Solutions are selected to be used as parents of the next generation of solutions. Typically, as many parents are chosen as there are members in the initial population. The chance that a solution will be chosen to be a parent is related to the results of the evaluation of that solution: better solutions are more likely to be chosen as parents. Usually, the better solutions are chosen as parents multiple times, so that they will be the parents of multiple new solutions, while the poorer solutions are not chosen at all.

(4) Pairing of parents: The parent solutions are formed into pairs. The pairs are often formed at random but in some implementations dissimilar parents are matched to promote diversity in the children.

(5) Generation of children: Each pair of parent solutions is used to produce two new children. Either a mutation operator is applied to each parent separately to yield one child from each parent or the two parents are combined using a recombination operator, producing two children which each have some similarity to both parents. To take the six-variable example, one simple recombination technique would be to have the solutions in each pair merely trade their last three variables, thus creating two new solutions (and the original parent solutions may be allowed to survive). Thus, a child population the same size as the original population is produced. The use of recombination operators is a key difference between genetic algorithms and other optimization or search techniques. Recombination operating generation after generation ultimately combines the “building blocks” of the optimal solution that have been discovered by successful members of the evolving population into one individual. In addition to recombination techniques, mutation operators work by making a random change to a randomly selected component of the parent.

(6) Rating of children: The members of the new child population are evaluated. Since the children are modifications of the better solutions from the preceding population, some of the children may have better ratings than any of the parental solutions.

(7) Combining the populations: The child population is combined with the original parent population to produce a hew population. One way to do this is to accept the best half of the solutions from the union of the child population and the source population. Thus, the total number of solutions stays the same but the average rating can be expected to improve if superior children were produced. Any inferior children that were produced will be lost at this stage. Superior children become the parents of the next generation.

(8) Checking for termination: If the program is not finished, steps 3 through 7 are repeated. The program can end if a satisfactory solution (i.e., a solution with an acceptable rating) has been generated. More often, the program is ended when either a predetermined number of iterations has been completed, or when the average evaluation of the population has not improved after a large number of iterations.

The present invention is directed to the application of genetic algorithms to HTS methodology, particularly for materials systems. Because the number of constraints for a materials system can be quite large, the number of combinations of constraints may be a very large number. In lieu of physical evaluation of each combination of constraints, a genetic algorithm is applied to a population of constraints to define a second population of constraints that is a generation of the first. The genetic algorithm then searches for favorable combinations of constraints to produce a materials system that meets specified criteria. The algorithm “short cuts” the investigatory process by avoiding exhaustive sequential population testing.

The invention can be applied to screen for a catalyst to prepare, e.g., a diaryl carbonate by carbonylation. Diaryl carbonates such as diphenyl carbonate can be prepared by reaction of hydroxyaromatic compounds such as phenol with oxygen and carbon monoxide in the presence of a catalyst composition comprising a Group VIIIB metal such as palladium or a compound thereof and a halide source such as a quaternary ammonium or hexaalkylguanidinium bromide.

Various methods for the preparation of diaryl carbonates by a carbonylation reaction of hydroxyaromatic compounds with carbon monoxide and oxygen have been disclosed. In general, the carbonylation reaction has required a rather complex catalyst. Reference is made, for example, to Chaudhari et al., U.S. Pat. No. 5,917,077. The catalyst compositions described therein comprise a Group VIIIB metal (i.e., a metal selected from the group consisting of ruthenium, rhodium, palladium, osmium, iridium and platinum) or a complex thereof. They are used in combination with a bromide source, as illustrated by tetra-n-butylammonium bromide and hexaethylguanidinium bromide.

Other catalytic constituents are necessary in accordance with Chaudhari et al. They include inorganic cocatalysts, typically complexes of cobalt(II) salts with organic compounds capable of forming complexes, especially pentadentate complexes, therewith. Illustrative organic compounds of this type are nitrogen-heterocyclic compounds including pyridines, bipyridines, terpyridines, quinolines, isoquinolines and biquinolines; aliphatic polyamines such as ethylenediamine and tetraalkylethylenediamines; crown ethers; aromatic or aliphatic amine ethers such as cryptanes; and Schiff bases. The especially preferred inorganic cocatalyst in many instances is a cobalt(II) complex with bis-3-(salicylalamino)propylmethylamine.

Chaudhari et al. also claim that organic cocatalysts are necessary. They may include various terpyridine, phenanthroline, quinoline and isoquinoline compounds including 2,2′:6′,2″-terpyridine, 4-methylthio-2,2′:6′,2″-terpyridine and 2,2′:6′,2″-terpyridine N-oxide, 1,10-phenanthroline, 2,4,7,8-tetramethyl- 1,10-phenanthroline, 4,7-diphenyl- 1,10, phenanthroline and 3,4,7,8-tetramethy- 1,10-phenanthroline. The terpyridines and especially 2,2′:6′,2″-terpyridine have generally been preferred.

Any hydroxyaromatic compound may be employed. Monohydroxyaromatic compounds, such as phenol, the cresols, the xylenols and p-cumylphenol are generally preferred with phenol being most preferred. The invention may, however, also be employed with dihydroxyaromatic compounds such as resorcinol, hydroquinone and 2,2-bis(4-hydroxyphenyl)propane or “bisphenol A,” whereupon the products are polycarbonates.

Another constituent of the Chaudhari catalyst composition is one of the Group VIIIB metals, preferably palladium, or a compound thereof. Thus, palladium black or elemental palladium deposited on carbon are suitable, as well as palladium compounds such as halides, nitrates, carboxylates, salts with aliphatic .beta.-diketones and complexes involving such compounds as carbon monoxide, amines, phosphines and olefins. Preferred in most instances are palladium(II) salts of organic acids, most often C₂₋₆aliphatic carboxylic acids and of ∃.-diketones such as 2,4-pentanedione. Palladium(II) acetate and palladium(II) 2,4-pentanedionate are generally most preferred.

The Chaudhari catalytic material also contains a bromide source. It may be a quaternary ammonium or quaternary phosphonium bromide or a hexaalkylguanidinium bromide. The guanidinium salts are often preferred; they include the ∀,Σ.-bis(pentaalkylguanidinium)alkane salts. Salts in which the alkyl groups contain 2-6 carbon atoms and especially tetra-n-butylammonium bromide and hexaethylguanidinium bromide are particularly preferred.

Another Chaudhari catalyst constituent is a polyaniline in partially oxidized and partially reduced form can be employed.

Other reagents in the method are oxygen and carbon monoxide, which react with the phenol to form the desired diaryl carbonate.

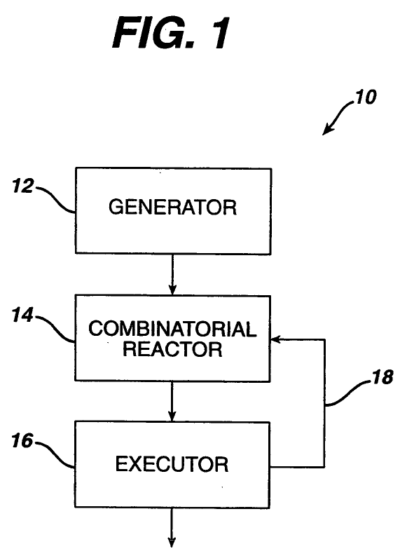

FIG. 1 is a schematic representation of an exemplary system for screening constructs to determine a problem solution. In FIG. 1, a system 10 includes a generator 12, a combinatorial reactor 14 and an executor 16. Generator 12 can be a controller, microprocessor, computer or calculator or code or any structure that can provide a binary string representing a random first population of the constructs.

Combinatorial reactor 14 can include a reaction vessel such as the combination of an array tray and reaction furnace or a continuous longitudinal reactor to synthesize each construct by a high throughput screening methodology referred to as COS in the field of organic chemistry. In the representation of FIG. 1, the reactor 14 includes an analyzer to determine a fitness function for each synthesized construct of the population. The analyzer can utilize chromatography, infra red spectroscopy, mass spectroscopy, laser mass spectroscopy, microspectroscopy, NMR or the like to determine a property or constituency of each construct.

Executor 16 can be a controller, microprocessor, computer or calculator or code or any structure that can execute genetic algorithms on the binary string representing a random first population of the constructs. Structurally, executor 16 can be a code of the same computer or microprocessor that includes a code according to the requirements of generator 12. The executor executes a genetic algorithm on the first population to produce a generation that defines a second population of constructs according to the invention. The second population can be then synthesized and analyzed by recycling 18 into combinatorial reactor 14.

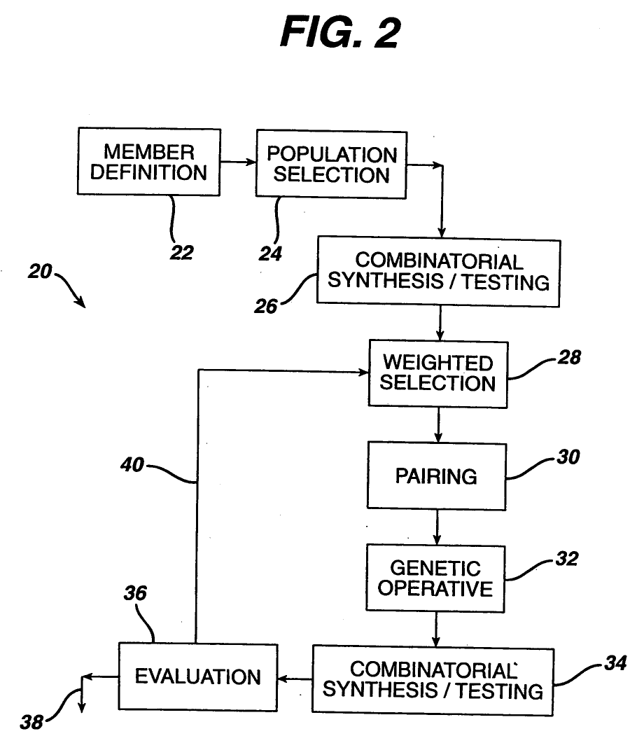

FIG. 2 is a schematic representation of a genetic algorithmic iterative high throughput screening method. In FIG. 2, a method 20 includes iterative steps of member definition 22, population selection 24, combinatorial synthesis/testing 26, weighted selection 28, pairing 30, genetic operation 32, combinatorial synthesis/testing 34 and evaluation 36. The genetic algorithmic iterative high throughput screening method 20 of FIG. 2 can be conducted, for example, in the system 10 of FIG. 1.

Referring to FIG. 2, in member definition step 22, parameters of an initial space can be determined and the parameters used to construct a genetic code that represents entities of a population. A sampling of the population can be randomly determined 24 and designated a first population. Each of the iterative steps 22 and 24 can be conducted by generator 12 of system 10 of FIG. 1.

Each entity of the first population can be synthesized and analyzed in combinatorial synthesis/testing step 26. This step can be conducted in combinatorial reactor 14 of system 10 of FIG. 1. Step 26 determines a property that can be used to evaluate each entity of the first population. For example, the property may be effectiveness as a catalyst or flame retardant or toxicity or rate of production or yield of a set of reaction parameters or any property of interest.

The combinatorial synthesis/testing step can be any suitable HTS method. For example, each of the first population of entities can be deposited in respective wells of an array; the population reacted to form a plurality of products and the property of each of the plurality of products detected by chromatography, infra red spectroscopy, mass spectroscopy, laser mass spectroscopy, microspectroscopy, NMR or the like. In another suitable method, a population of entities is synthesized by providing a first reactant system at least partially embodied in a liquid and contacting the liquid with a second reactant system at least partially embodied in a gas, the second reactant system having a mass transport rate into the liquid wherein the liquid forms a film having a thickness sufficient to allow a reaction rate that is essentially independent of the mass transport rate of the second reactant system into the liquid.

In step 28, each entity of the first population can be weighted according to the property determined in step 26 and a selection of entities is made from the weighted first population. Each entity of the selection can be paired 30 with another entity. A genetic operative can then executed 32 on each set of paired entities to produce children or a second generation of entities. Step 32 represents application of a recombination operator to the data representations. Recombination operators include crossover, single point crossover, swap crossover, uniform random crossover and the like. A “uniform random crossover” is a genetic algorithmic operator that exchanges parameters at randomly selected corresponding loci of paired population members. For example, if the operator determines that crossover should occur at loci 2 and 6 of paired members [A,A,A,A,A,A,A,A] and [B,B,B,B,B,B,B,B], it produces children members [A,B,A,A,A,B,A,A] and [B,A,B,B,B,A,B,B].

Each entity of the second population can then be synthesized and analyzed in the combinatorial synthesis/testing step 34. This step can be conducted in combinatorial reactor 14 of system 10 of FIG. 1. Step 34 determines the same property for the second population as was determined and used to evaluate each entity of the first population. The data for the second population can be used to designate a fit solution in an evaluation step 36 and the method can be terminated 38. Or the data can be recycled 40 to the weighted selection step 28 and the process repeated for any number of iterations to provide a most fit solution.

Each combinatorial syntheses/testing step of FIG. 2 can be carried out in combinatorial reactor 14 of system 10. Similarly, the other steps of method 20 can be carried out in generator 12 or executor 16 of system 10 as the case may be.

The following example is included to provide additional guidance to those skilled in the art in practicing the claimed invention. The example provided is merely representative of the work that contributes to the teaching of the present application. Accordingly, the example is not intended to limit the invention, as defined in the appended claims, in any manner.

### EXAMPLE

This example illustrates the identification of an active and selective catalyst for the production of aromatic carbonates. The procedure identifies the best catalyst from within a complex chemical space, where the chemical space is defined as an assemblage of all possible experimental conditions defined by a set of variable parameters such as formulation ingredient identity or amount. In the specific instance, the experimental formulation consists of six chemical species shown in TABLE 2.

TABLE 2

| | Type | Amount |
|---|---|---|
| | parameter variation | Parameter variation |
| Precious metal catalyst | Held Constant | Held constant |
| (PC) | | |
| Metal Catalyst 1 (M1) | Chosen (without | Each varied |
| Metal Catalyst 2 (M2) | replacement) | independently in |
| | from a set | amount. Possible |
| | of 22 | values were |
| Metal Catalyst 3 (M3) | possible metal | 2, 4, 6, 8, 10 |
| | compounds | (as molar ratios to |
| | | precious metal |
| | | catalyst) |
| Cosolvent (CS) | Chosen from two | Varied independently |
| | possible solvents | in amount. Possible |
| | | values were 500, 1500, |
| | | 4000 (as molar ratios |
| | | to precious metal |
| | | catalyst) |
| Hydroxyaromatic | Held constant | Sufficient added to |
| compound | | achieve constant |
| | | sample volume |

The size of an initial chemical space defined by the parameters of TABLE 2 is calculated as 1,155,000 possibilities. Conventional screening techniques can not be-practically used to select a best system because of the large size of the chemical space. Hence, the size is screened by a genetic algorithm technique according to the invention.

The population of potential solutions is composed into the linked list abbreviated in TABLE 3. Eight loci positions are defined for each member of a first population of entities. Each locus position represents one of the chemical identifiers of TABLE 3. A determination is made to define a population of 100 members each represented by one of the eight loci formulations. This population is chosen to be large enough to ensure that at least 55 unique members without duplicate M1/M2/M3's are generated. Each locus of the 100 members is chosen by application of the randomization functionality of EXCEL® & software available from Microsoft Corporation. The first 100 member population is then examined manually and identical members and members that have duplicate M1, M2 or M3 metals are manually eliminated. Fifty-five members are selected randomly from the remaining formulations to give the 110 duplicate runs required to fit an available experimental apparatus.

TABLE 3

| Position | Chemical Identifier | Possible Values |
|---|---|---|
| 1 | M1 | 1-22 |
| 2 | M1:PC ratio | 2, 4, 6, 8, 10 |
| 3 | M2 | 1-22 |
| 4 | M2:PC ratio | 2, 4, 6, 8, 10 |
| 5 | M3 | 1-22 |
| 6 | M3:PC ratio | 2, 4, 6, 8, 10 |
| 7 | CS | 1, 2 |
| 8 | CS:PC ratio | 500, 1500, 4000 |

In this example, the precious metal is palladium; the 22 metal compounds chosen as cocatalysts (M1, M2, M3) are acetylacetonates of Fe, Cu, Ce, Yb, Eu, Mn, Co, Bi, Ni, Zn, TiO, Cr, Ir, Ru, Rh, Ga, Cd, Ca, Re, In, Cs and La. Cosolvents (CS) are dimethylacetamide (DMAA) and dimethylformamide (DMFA) and the hydroxyaromatic compound is phenol.

The selected members are synthesized in duplicate for a total of 110 actual experiments. The members are evaluated for performance in a process for the production of aromatic carbonates. In this process, In the evaluation, each of the metal acetylacetonates, the DMAA, and the DMFA are made up as stock solutions in phenol. Appropriate quantities of each stock solution are then combined using a Hamilton MicroLab 4000™ laboratory robot into a single vial for mixing. For example, to produce mix 1 of TABLE 4, the stock solutions are 0.01 molar Pd(acetylacetonate), 0.01 molar each of Cr(acetylacetonate), Ca(acetylacetonate) and Gd(acetylacetonate) and 10 molar DMFA. Ten ml of each stock solution are produced by manual weighing and mixing. Aliquots of the stock solutions are measured as follows in TABLE 4. The mixture is stirred using a miniature magnetic stirrer, and then 25 microliters are measured out using the Hamilton robot to each of two 2-ml vials. This small quantity forms a thin film on the vial bottom.

TABLE 4

| | |
|---|---|
| 0.01 molar Pd(acetylacetonate) | 25 microliters |
| 0.01 molar Cr(acetylacetonate) | 50 microliters |
| 0.01 molar Ca(acetylacetonate) | 75 microliters |
| 0.01 molar Gd(acetylacetonate) | 225 microliters |
| 10 molar DMFA | 37.5 microliters |
| Pure phenol | 601 microliters |

After each mixture is made, mixed, and distributed to the 2-ml vials, the vials are capped using “star” caps (which allow gas exchange with the environment) and placed in a holder that fits precisely into a 1 gallon Autoclave Engineers high pressure autoclave. The autoclave is pressurized with an 8% mixture of oxygen in carbon monoxide at 100 bar, heated to 100° C. over a 45 minute period and then held at 100C three hours. It is then returned to room temperature in 45 minutes, depressurized and the vials removed and the products analyzed using gas chromatography.

Performance is expressed numerically as a catalyst turnover number or TON. TON is defined as the number of moles of aromatic carbonate produced per mole of Palladium catalyst charged. Duplicate experiments are averaged to give an average TON. The results are shown in TABLE 5.

TABLE 5

| | | | | | | | | | | ave | Probability of |
|---|---|---|---|---|---|---|---|---|---|---|---|
| | Mix | M1 | M1:Pd | M2 | M2:Pd | M3 | M3:Pd | CS | CS:Pd | TON | Selection |
| 1 | 48 | Ca | 1 | Cu | 9 | Cd | 7 | DMAA | 4000 | 5810 | 12.50% |
| 2 | 47 | Cd | 4 | Ca | 6 | Cu | 5 | DMAA | 1500 | 5730 | 12.33% |
| 3 | 31 | Fe | 1 | Cu | 10 | Ni | 2 | DMAA | 1500 | 4560 | 9.81% |
| 4 | 35 | Fe | 6 | Cu | 5 | TiO | 10 | DMAA | 4000 | 2960 | 6.37% |
| 5 | 13 | Fe | 7 | In | 3 | Cd | 9 | DMFA | 4000 | 1740 | 3.74% |
| 6 | 6 | Mn | 4 | Ca | 9 | Cr | 2 | DMAA | 500 | 1560 | 3.38% |
| 7 | 23 | Mn | 9 | Ca | 1 | Gd | 5 | DMFA | 4000 | 1530 | 3.29% |
| 8 | 39 | Zn | 8 | Mn | 6 | Fe | 5 | DMAA | 4000 | 1470 | 3.16% |
| 9 | 52 | Mn | 9 | Ni | 1 | Cd | 10 | DMAA | 4000 | 1470 | 3.16% |
| 10 | 22 | Ir | 3 | Ni | 2 | TiO | 8 | DMAA | 500 | 1470 | 3.16% |
| 11 | 42 | In | 10 | Eu | 10 | Ir | 9 | DMFA | 500 | 1420 | 3.06% |
| 12 | 30 | In | 4 | Gd | 9 | Cd | 7 | DMFA | 1500 | 1400 | 3.01% |
| 13 | 34 | Co | 8 | Fe | 7 | Eu | 2 | DMFA | 1500 | 1390 | 2.99% |
| 14 | 18 | In | 8 | Re | 4 | La | 3 | DMFA | 500 | 1290 | 2.78% |
| 15 | 45 | Cs | 10 | Zn | 6 | Ce | 6 | DMFA | 500 | 910 | 1.96% |
| 16 | 18 | Bi | 4 | Ce | 8 | Eu | 10 | DMFA | 500 | 880 | 1.89% |
| 17 | 26 | TiO | 9 | Ru | 3 | Zn | 9 | DMFA | 1500 | 820 | 1.76% |
| 18 | 38 | Cs | 5 | Re | 4 | Fe | 10 | DMAA | 500 | 780 | 1.68% |
| 19 | 36 | Zn | 4 | Re | 5 | Cs | 2 | DMFA | 500 | 670 | 1.44% |
| 20 | 29 | La | 3 | Bi | 2 | Yb | 3 | DMFA | 500 | 660 | 1.42% |
| 21 | 53 | Ce | 1 | Yb | 8 | Cs | 6 | DMFA | 4000 | 630 | 1.36% |
| 22 | 4 | Ir | 5 | Cd | 8 | Fe | 2 | DMAA | 500 | 610 | 1.31% |
| 23 | 10 | Eu | 7 | Zn | 6 | Gd | 5 | DMFA | 500 | 580 | 1.25% |
| 24 | 44 | Ni | 1 | Yb | 4 | Cs | 5 | DMFA | 1500 | 490 | 1.05% |
| 25 | 17 | La | 7 | Eu | 1 | Ce | 1 | DMFA | 4000 | 460 | 0.99% |
| 26 | 33 | Re | 2 | La | 1 | Cd | 3 | DMFA | 4000 | 450 | 0.97% |
| 27 | 11 | Bi | 5 | Yb | 2 | Cr | 4 | DMFA | 4000 | 440 | 0.95% |
| 28 | 3 | Eu | 1 | Gd | 7 | Ca | 10 | DMFA | 4000 | 430 | 0.93% |
| 29 | 46 | Fe | 3 | Ru | 2 | Ce | 7 | DMFA | 1500 | 410 | 0.88% |
| 30 | 50 | Ca | 6 | Cd | 1 | La | 1 | DMFA | 1500 | 390 | 0.84% |
| 31 | 21 | $$ | 9 | La | 1 | Cs | 2 | DMFA | 4000 | 370 | 0.80% |
| 32 | 1 | $$ | 2 | Ca | 3 | Gd | 9 | DMFA | 500 | 360 | 0.77% |
| 33 | 40 | Rh | 10 | Co | 8 | Mn | 10 | DMAA | 1500 | 360 | 0.77% |
| 34 | 8 | Ir | 1 | Rh | 7 | Yb | 4 | DMFA | 500 | 350 | 0.75% |
| 35 | 49 | Cd | 10 | Cs | 1 | Bi | 5 | DMAA | 500 | 340 | 0.73% |
| 36 | 12 | Fe | 2 | In | 2 | Ce | 6 | DMAA | 1500 | 320 | 0.69% |
| 37 | 43 | Cr | 3 | Rh | 4 | Mn | 1 | DMFA | 500 | 300 | 0.65% |
| 38 | 54 | Co | 8 | Yb | 9 | Ir | 7 | DMFA | 4000 | 240 | 0.52% |
| 39 | 32 | Re | 3 | Cs | 3 | Ni | 2 | DMFA | 500 | 190 | 0.41% |
| 40 | 24 | Eu | 2 | Cd | 2 | Fe | 5 | DMAA | 1500 | 100 | 0.22% |
| 41 | 37 | Ca | 9 | Cu | 4 | La | 1 | DMAA | 4000 | 90 | 0.19% |
| 42 | 14 | Bi | 9 | In | 3 | Ru | 5 | DMFA | 500 | 40 | 0.09% |
| 43 | 2 | Rh | 6 | Cs | 6 | Gd | 7 | DMAA | 4000 | 0 | 0.00% |
| 44 | 5 | Co | 1 | Ru | 2 | Zn | 6 | DMAA | 500 | 0 | 0.00% |
| 45 | 7 | Cd | 4 | Ru | 5 | Fe | 10 | DMAA | 4000 | 0 | 0.00% |
| 46 | 9 | Bi | 7 | Mn | 3 | Ru | 7 | DMFA | 500 | 0 | 0.00% |
| 47 | 15 | Re | 2 | Ni | 9 | Zn | 4 | DMAA | 4000 | 0 | 0.00% |
| 48 | 19 | Yb | 4 | TiO | 6 | Mn | 4 | DMFA | 4000 | 0 | 0.00% |
| 49 | 20 | Ca | 1 | Yb | 7 | Bi | 3 | DMAA | 4000 | 0 | 0.00% |
| 50 | 25 | Rh | 2 | Gd | 10 | La | 2 | DMAA | 1500 | 0 | 0.00% |
| 51 | 27 | Re | 7 | Gd | 3 | Co | 1 | DMAA | 4000 | 0 | 0.00% |
| 52 | 28 | Bi | 10 | Mn | 5 | Ru | 10 | DMFA | 1500 | 0 | 0.00% |
| 53 | 41 | Rh | 10 | Cr | 6 | Ca | 8 | DMAA | 4000 | 0 | 0.00% |
| 54 | 51 | Yb | 9 | Ru | 6 | Rh | 4 | DMAA | 500 | 0 | 0.00% |
| 55 | 55 | Cr | 7 | Ir | 9 | In | 7 | DMAA | 1500 | 0 | 0.00% |
| | | | | | | | | | Total | 46470 | |
| | | | | | | | | | TON | | |

One hundred and ten (110) members are computer selected from the 55 formulations generated in the initialization. The members are chosen in proportion to TON: probability of selection=member TON/Total TON. As a result, formulations representing better solutions (higher TON) are chosen multiple times. For example, the formulation of Row 1 of TABLE 5 represents a 15.4% probability of selection. Since that probability is applied for each of the 110 selections, probability calculations estimate that the most likely number of times a member of row 1 will be selected is 16 to 18 (110×0.1538=16.92). This formulation is selected 17 times as a parent. Similarly, the most likely number of times the formulation in row 28 would be selected is estimated to be one (110×0.009=0.99).

The 110 parents are paired by computer using a random genetic algorithm program to provide 55 pairs that are used as parents. The program randomly selects two members from the population without replacement and enters them into a list as pairs.

A uniform random crossover operator is applied by computer using a genetic algorithm program to each pair of parents to produce two children members for each pair. In this example, the operator is modified to avoid duplication of metal elements in a single solution as follows: The paired members are detected to determine if crossover will cause duplication in a child. If a chance of duplication is determined, then the metal elements are reordered in a parent of the pair so that the duplication is prevented. For example, if the pair A[Cu,6,Ca,4,Fe,10,DMFA,500] and B[Ca,2,Fe,8,Cr,2,DMAA,1500] is detected, the operator will reorder parent B to [Cr,2,Ca,2,Fe,8,DMAA,1500] to prevent duplication upon crossover.

The crossover operator with detection and duplication prevention generates 110 solutions as children. Several duplicates are observed. A first 55 valid and unique individuals in the list are selected and evaluated for TON performance.

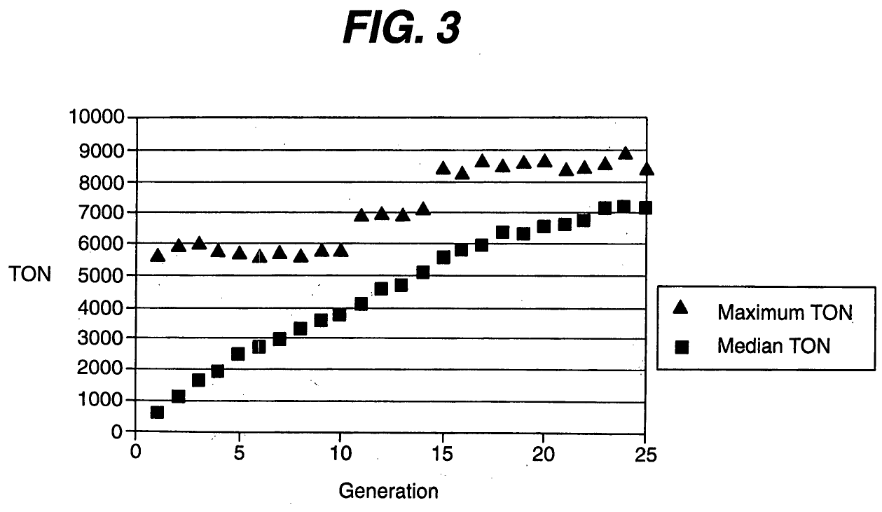

The procedures of selection, pairing, crossover and evaluation are repeated as described above for a total of 25 cycles. Results at the end of 25 generations are shown in FIG. 3. FIG. 3 shows several jumps in the maximum TON as the genetic algorithm succeeds in locating increasingly favorable combinations of the process parameters. At the end of the process, the population is found to have a large fraction of its members with Fe, La, and Mn as the metals and DMAA as the cosolvent. Further investigation by conventional means confirms that GA selects the optimum system of TABLE 6.

TABLE 6

| Component | Ratio: Pd |
|---|---|
| Fe | 10 |
| La | 8 |
| Mn | 4 |
| DMNA | 500 |

It will be understood that each of the elements described above, or two or more together, may also find utility in applications differing from the types described herein. While the invention has been illustrated and described as embodied in a high throughput screening method and system, it is not intended to be limited to the details shown, since various modifications and substitutions can be made without departing in any way from the spirit of the present invention. For example, additional HTS methodology can be used in concert with the disclosed examples. As such, further modifications and equivalents of the invention herein disclosed may occur to persons skilled in the art using no more than routine experimentation, and all such modifications and equivalents are believed to be within the spirit and scope of the invention as defined by the following claims.

What is claimed is:

1. A method, comprising steps of:
(A) synthesizing a first population of entities and detecting a property of each of said entities by a high throughput screening (HTS) method and
(B) executing a genetic algorithm based on said property of said entities to identify a second population of entities.

2. The method of claim 1, wherein said step (B) comprises at least one operation selected from (i) mutation, (ii) crossover, (III) mutation and selection (iv) crossover and selection and (v) mutation, crossover and selection.

3. The method of claim 1, comprising randomly identifying said first population of entities prior to synthesizing said first population according to step (A).

4. The method of claim 1, further comprising generating a binary string representing said first population of entities and step (B) comprises executing a genetic algorithm with a processor on said binary string to produce a binary string representing said second population of entities.

5. The method of claim 1, further comprising generating a binary string representing variable parameters of said first population of entities and step (B) comprises executing a genetic algorithm with a processor on said binary string to produce a binary string representing said second population of entities.

6. The method of claim 1, further comprising generating a binary string representing variable parameters of entities, synthesizing said entities and selecting said first population from said entities and step (B) comprises executing a genetic algorithm with a processor on said binary string to produce a binary string representing said second population of entities.

7. The method of claim 1, further comprising generating a binary string representing variable parameters of entities, synthesizing said entities, evaluating said synthesized entities for a desired property, weighting said entities according to an hierarchy of fitness of said property and selecting said first population as a sampling from said weighed entities and step (B) comprises executing a genetic algorithm with a processor on said binary string to produce a binary string representing said second population of entities.

8. The method of claim 1, further comprising generating a binary string representing variable parameters of entities, synthesizing said entities, evaluating said synthesized entities for a desired property, pairing said entities and (B) comprises executing a genetic algorithm with a processor on said binary string to produce a binary string representing said second population of entities.

9. The method of claim 1, further comprising generating a binary string representing variable parameters of entities, synthesizing said entities, evaluating said synthesized entities for a desired property and pairing said entities and (B) comprises executing a genetic algorithm comprising a uniform random crossover operator to produce a binary string representing said second population of entities.

10. The method of claim 1, further comprising generating a binary string representing variable parameters of entities, synthesizing said entities, evaluating said synthesized entities for a desired property, weighting said entities according to an hierarchy of fitness according to said property, selecting said first population as a sampling from said weighed entities and pairing said entities and step (B) comprises executing a genetic algorithm with a processor on said binary string to produce a binary string representing said second population of entities.

11. The method of claim 1, further comprising conducting steps (A) and (B) on said second population of entities to produce a third population of entities.

12. The method of claim 1, further comprising repeating steps (A) and (B) on said second population of entities and subsequent populations of entities until a fit entity is identified.

13. The method of claim 1, wherein said first population of entities is synthesized by steps of:
providing a first reactant system at least partially embodied in a liquid; and
contacting the liquid with a second reactant system at least partially embodied in a gas, the second reactant system having a mass transport rate into the liquid wherein the liquid form is a film having a thickness sufficient to allow a reaction rate that is essentially independent of the mass transport rate of the second reactant system into the liquid to synthesize said first population of entities.

14. The method of claim 1, further comprising synthesizing said second population of entities by steps of.
providing a first reactant system at least partially embodied in a liquid; and
contacting the liquid with a second reactant system at least partially embodied in a gas, the second reactant system having a mass transport rate into the liquid wherein the liquid forms a film having a thickness sufficient to allow a reaction rate that is essentially independent of the mass transport rate of the second reactant system into the liquid to synthesize said send population of entities.

15. The method of claim 1, wherein said HTS method is acombinatorial organic synthesis (COS).

16. The method of claim 1, wherein said first population of entities is a catalyst system.

17. The method of claim 1, wherein said first population of entities is a catalyst system comprising a Group VIII B metal.

18. The method of claim 1, wherein said first population of entities is a catalyst system comprising palladium.

19. The method of claim 1, wherein said first population of entities is a catalyst system comprising a halide composition.

20. The method of claim 1, wherein said first population of entities is a catalyst system that includes an inorganic co-catalyst.

21. The method of claim 1, wherein said first population of entities is a catalyst system that includes a combination of inorganic co-catalysts.

22. A high throughput screening (HTS) method, comprising:
(A) depositing each of a first population of entities in respective wells of an array;
(B) reacting said population to form a plurality of products;
(C) detecting a property of each of said plurality of products; and
(D) executing a genetic algorithm based on said property of said plurality of products to identify a second population of entities.

23. The method of claim 22, further comprising:
(E) depositing each of said second population of entities in respective wells of an array; and
(F) reacting said second population to form a second plurality of products.

24. The method of claim 22, comprising randomly identifying said first population of entities prior to depositing said first population according to step (A).

25. The method of claim 22, wherein said step (D) comprises an at least one operation selected from (i) mutation, (ii) crossover, (III) mutation and selection (iv) crossover and selection and (v) mutation, crossover and selection.

26. The method of claim 22, further comprising generating a binary string representing said first population of entities and step (D) comprises executing a genetic algorithm with a processor on said binary string to produce a binary string representing said second population of entities.

27. The method of claim 22, wherein said HTS method is a combinatorial organic synthesis (COS).

28. The method of claim 22, wherein said first population of entities is a catalyst system.

29. The method of claim 22, wherein said first population of entities is a catalyst system comprising a Group VIII B metal.

30. The method of claim 22, wherein said first population of entities is a catalyst system comprising palladium.

31. The method of claim 22, wherein said first population of entities is a catalyst system comprising a halide composition.

32. The method of claim 22, wherein said first population of entities is a catalyst system that includes an inorganic co-catalyst.

33. The method of claim 22, wherein said first population of entities is a catalyst system that includes a combination of inorganic co-catalysts.

34. A method for preparing a diaryl carbonate which comprises contacting at least one hydroxyaromatic compound with oxygen and carbon monoxide in the presence of an amount effective for carbonylation of at least one catalyst composition comprising a Group VIIIB metal or a compound thereof, a bromide source and a polyaniline wherein said catalyst composition is selected according to a genetic algorithm screening process.

35. The method of claim 34, wherein at one of said Group VIIIB metal or compound thereof, said bromide source and said polyaniline is selected by said genetic algorithm screening process.

36. The method of claim 34, wherein a concentration of at least one of said Group VIIIB metal or compound thereof, said bromide source and said polyaniline is selected by said genetic algorithm screening process.

37. The method of claim 34, wherein said Group VIIIB metal or compound thereof, said bromide source and said polyaniline are selected by said genetic algorithm screening process.

38. The method of claim 34, wherein concentrations of said Group VIIIB metal or compound thereof, said bromide source and said polyaniline are selected by said genetic algorithm screening process.

39. The method of claim 34, wherein said Group VIIIB metal or compound thereof, said bromide source and said polyaniline are selected by said genetic algorithm screening process and concentrations thereof are selected by said algorithm screening process.

40. A method of selecting a carbonylation catalyst, comprising:
(A) synthesizing a first population of prospective carbonylation catalyst entities and detecting a property of each of said entities; and
(B) executing a genetic algorithm based on said property of said entities to identify a second population of prospective carbonylation catalyst entities.

41. A system for screening constructs to determine a problem solution, comprising:
a generator to provide a binary string representing a random first population of said constructs;
a combinatorial reactor to synthesize each construct according to said representation of said first population and to determine a fitness function for each construct of said population by a high throughput screening process; and
an executor to execute a genetic algorithm on said first population to produce a generation that defines a second population of said materials.
